(19) United States
(12) Patent Application Publication
(10) Pub. No.: US 2012/0209309 A1
(43) Pub. Date: Aug. 16, 2012

(54) VASO-OCCLUSIVE DEVICE

(75) Inventors: Hancun CHEN, San Ramon, CA (US); Richard MURPHY, Sunnyvale, CA (US); Jimmy DAO, San Jose, CA (US)

(73) Assignees: Stryker NV Operations Limited, Dublin (IE); Stryker Corporation, Kalamazoo, MI (US)

(21) Appl. No.: 13/369,211

(22) Filed: Feb. 8, 2012

Related U.S. Application Data
(60) Provisional application No. 61/442,107, filed on Feb. 11, 2011.

(51) Int. Cl. A61M 29/00

(52) U.S. Cl. 606/194

(57) ABSTRACT

A vaso-occlusive device includes a first wire having a first cross-sectional geometry and a second wire having a second cross-sectional geometry, where the first cross-sectional geometry is different from the second cross-sectional geometry. The first wire may be wound to form a first coil, where the second wire is wound to form a second coil defining a lumen therein, and where the first coil is disposed at least partially in the lumen. The first wire and the second wire may be co-wound to form a single coil.

### RELATED APPLICATION DATA

The present application claims the benefit under 35 U.S.C. §119 to U.S. Provisional Application No. 61/442,107, filed Feb. 11, 2011, the contents of which are hereby incorporated herein by reference as though set forth in full.

### FIELD

The field of the disclosed inventions generally relates to vaso-occlusive devices for establishing an embolus or vascular occlusion in a vessel of a human or veterinary patient. More particularly, the disclosed inventions relate to vaso-occlusive coils.

### BACKGROUND

Vaso-occlusive devices or implants are used for a wide variety of reasons, including treatment of intra-vascular aneurysms. Commonly used vaso-occlusive devices include soft, helically wound coils formed by winding a platinum (or platinum alloy) wire strand about a “primary” mandrel. The coil is then wrapped around a larger, “secondary” mandrel, and heat treated to impart a secondary shape. For example, U.S. Pat. No. 4,994,069, issued to Ritchart et al., which is fully incorporated herein by reference, describes a vaso-occlusive device that assumes a linear, helical primary shape when stretched for placement through the lumen of a delivery catheter, and a folded, convoluted secondary shape when released from the delivery catheter and deposited in the vasculature.

In order to deliver the vaso-occlusive devices to a desired site in the vasculature, e.g., within an aneurysmal sac, it is well-known to first position a small profile, delivery catheter or “micro-catheter” at the site using a steerable guidewire. Typically, the distal end of the micro-catheter is provided, either by the attending physician or by the manufacturer, with a selected pre-shaped bend, e.g., 45°, 26°, “J”, “S”, or other bending shape, depending on the particular anatomy of the patient, so that it will stay in a desired position for releasing one or more vaso-occlusive device(s) into the aneurysm once the guidewire is withdrawn. A delivery or “pusher” wire is then passed through the micro-catheter, until a vaso-occlusive device coupled to a distal end of the delivery wire is extended out of the distal end opening of the micro-catheter and into the aneurysm. Once in the aneurysm, the vaso-occlusive devices bend to allow more efficient and complete packing The vaso-occlusive device is then released or “detached” from the end delivery wire, and the delivery wire is withdrawn back through the catheter. Depending on the particular needs of the patient, one or more additional occlusive devices may be pushed through the catheter and released at the same site.

One well-known way to release a vaso-occlusive device from the end of the pusher wire is through the use of an electrolytically severable junction, which is a small exposed section or detachment zone located along a distal end portion of the pusher wire. The detachment zone is typically made of stainless steel and is located just proximal of the vaso-occlusive device. An electrolytically severable junction is susceptible to electrolysis and disintegrates when the pusher wire is electrically charged in the presence of an ionic solution, such as blood or other bodily fluids. Thus, once the detachment zone exits out of the catheter distal end and is exposed in the vessel blood pool of the patient, a current applied through an electrical contact to the conductive pusher wire completes an electrolytic detachment circuit with a return electrode, and the detachment zone disintegrates due to electrolysis.

### SUMMARY

In one embodiment of the disclosed inventions, a vaso-occlusive device includes a first wire having a first cross-sectional geometry and a second wire having a second cross-sectional geometry, where the first cross-sectional geometry is different from the second cross-sectional geometry. In one such embodiment, the first wire is wound to form a first coil, where the second wire is wound to form a second coil defining a lumen therein, and where the first coil is disposed at least partially in the lumen. Optionally, one of the first and second coils is wound in a clockwise direction and the other of the first and second coils is wound in a counter-clockwise direction. In another such embodiment, the first wire and the second wire are co-wound to form a single coil. Optionally, the second wire forms more than one loop for each loop formed by the first wire. Optionally, the device also includes discrete contact points between the first coil and the second coil. Optionally, the device also includes a third wire at least partially co-wound with the first and second wires.

In some embodiments, the first wire has a first cross-sectional area, where the second wire having a second cross-sectional area, and where the first cross-sectional area is different from the second cross-sectional area. Optionally, the first wire is made from a first material, where the second wire is made from a second material, and where the first material is different from the second material. Optionally, at least one of the first and second wires may be made from a plurality of wires that are twisted together.

In another embodiment of the disclosed inventions, a vaso-occlusive device includes a coil wire having an un-flattened section, a first flattened section, and a second flattened section, where a short cross-sectional axis of the first flattened section lies substantially perpendicular to a longitudinal axis of the vaso-occlusive device, and a short cross-sectional axis of the second flattened section lies substantially parallel to the longitudinal axis of the vaso-occlusive device. Optionally, the un-flattened section of the coil wire has a triangular cross-sectional geometry.

Other and further aspects and features of embodiments of the disclosed inventions will become apparent from the ensuing detailed description in view of the accompanying figures.

### BRIEF DESCRIPTION OF THE DRAWINGS

The drawings illustrate the design and utility of embodiments of the disclosed inventions, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments and are not therefore to be considered limiting of its scope.

FIGS. 1-8 and 13 are detailed longitudinal cross-section views of vaso-occlusive devices constructed according to various embodiments of the disclosed inventions.

FIGS. 9 and 12 are detailed side views of vaso-occlusive devices according to respective embodiments of the disclosed inventions.

FIG. 10 is a detailed top view of the coil wire from which the vaso-occlusive device of FIG. 13 is made.

FIG. 11 is a detailed side view of the coil wire of FIG. 10.

FIG. 14 is a perspective view of a vaso-occlusive device in a natural state mode, illustrating one exemplary secondary configuration according to an embodiment of the disclosed inventions.

### DETAILED DESCRIPTION OF THE ILLUSTRATED EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments.

They are not intended as an exhaustive description of the invention or as a limitation on the scope of the invention, which is defined only by the appended claims and their equivalents. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

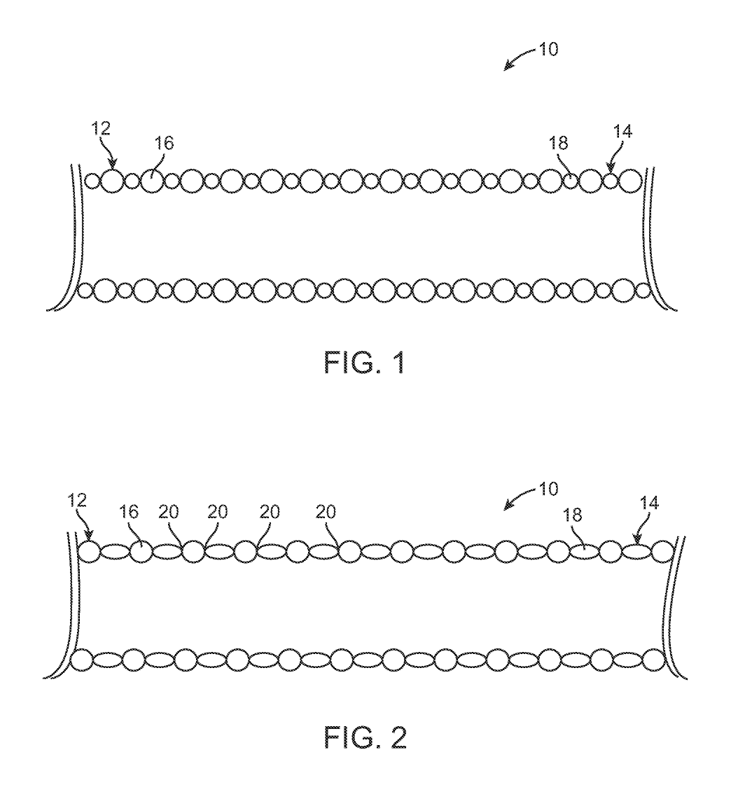

FIG. 1 illustrates a vaso-occlusive device 10 in accordance with one embodiment. The vaso-occlusive device 10 includes a first coil 12 and a second coil 14. The first and second coils 12, 14 each have a plurality of loops. The first and second coils 12, 14 are also co-wound together, i.e., loops of the first and second coils 12, 14 are collated as they are wound together to form the vaso-occlusive device 10. When first and second coils 12, 14 are collated together, as they are in FIG. 1, they form a single coil.

The first and second coils 12, 14 are made from the any suitable biocompatible material. For example, the first and second coils 12, 14 may be made from a metal, such as pure platinum. In other embodiments, the coils 12, 14 may be made from an alloy, such as platinum-tungsten alloy, e.g., 8% tungsten and the remainder platinum. In further embodiments, the coils 12, 14 may be made from platinum-iridium alloy, platinum rhenium alloy, or platinum palladium alloy. In still other embodiments, the coils 12, 14 may be made from biopolymers or bio-ceramic materials. Further, the first coil 12 may be made from a different material than the second coil 12.

In the illustrated embodiments, the first coil wire 16 from which the first coil 12 is made and the second coil wire 18 from which the second coil 14 is made have different outside diameters (“OD”). Because the bending moment of a coil is exponentially proportional to the diameter of the wire from which it is wound to the power of four, the vaso-occlusive device 10 has non-uniform bending behavior along its length and more readily bends at the second coil 14 loops along its length. These bending points provide better conformability and packing performance.

The ODs of the first and second coil wires 14, 16 can be optimized based on the target application of the vaso-occlusive device, i.e. framing, filling, or finishing. The larger ODs contribute to overall coil stiffness and the smaller ODs lead to easier bending. The ratio of the first coil wire 16 OD to the second coil wire 18 OD can also be optimized based on the target application of the vaso-occlusive device. In the vaso-occlusive device 10 in FIG. 1, the first coil wire 16 has an OD of 0.00125 inches and the second coil wire 18 has an OD of 0.001 inches. The first and second coil wires 16, 18 are Pt/8% W (platinum/tungsten) wires that are co-wound on a 0.007″ mandrel.

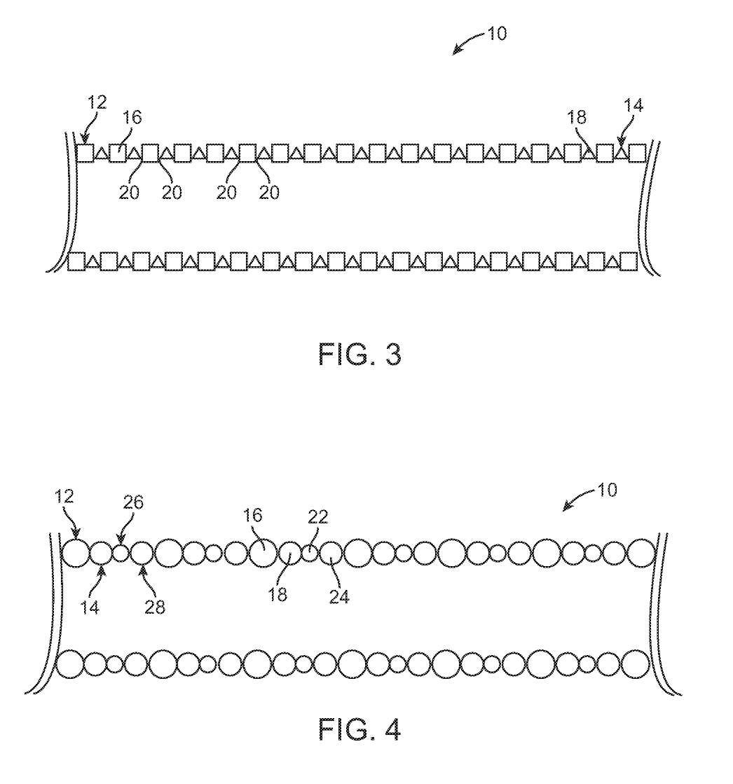

FIGS. 2 and 3 illustrate vaso-occlusive devices 10 formed by co-winding wires having different ODs and different cross-sectional geometries. In FIG. 2, the first coil wire 16 has a circular cross-sectional geometry, and the second coil wire 18 has an oval cross-sectional geometry. In FIG. 3, the first coil wire 16 has a square cross-sectional geometry, and the second coil wire 18 has a triangular cross-sectional geometry. In the vaso-occlusive devices 10 depicted in FIGS. 2 and 3, the first coil wires 16 have a larger OD than the second coil wires 18. The different cross-sectional geometries form more discrete contact points 20 between two adjacent loops, where the surface of at least one loop forms an acute angle. The more discrete contact points 20, in turn, result in easier bending when force is applied to the vaso-occlusive device 10. More generally, the first coil wire 16 and the second coil wire 18 may have different cross-sectional areas, particularly when they have different cross-sectional geometries.

FIG. 4 shows a vaso-occlusive device 10 formed by co-winding four coil wires having three different ODs. The first coil wire 16 has a larger OD. The second coil wire 18 and fourth coil wire 24 have the same or similar medium OD. The third coil wire 22 has a smaller OD. The presence of coil wires 16, 18, 22, 24 having large, medium, and smaller ODs allows more uniform and gradual bending of the vaso-occlusive device 10. The third coil wire 22 forms a third coil 26, and the fourth coil wire 24 forms a fourth coil 28.

Alternatively, the vaso-occlusive device 10 in FIG. 4 can be formed by co-winding three coil wires 16, 18, 22 having three different ODs, but forming two loops with the second coil wire 18 for every loop of the first and third coil wires 16, 22. The loop pattern in such a vaso-occlusive device repeats the following sequence: first coil loop; second coil loop; third coil loop; and second coil loop.

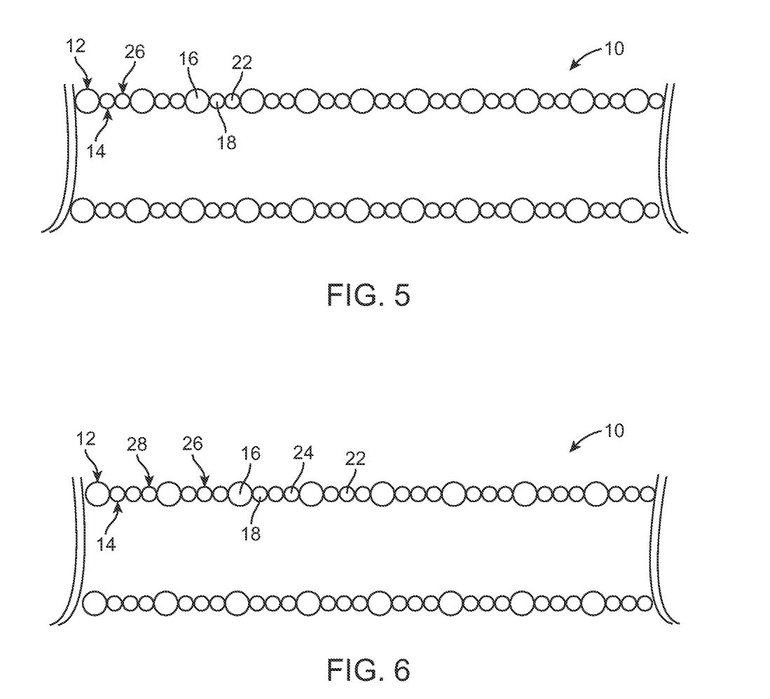

FIG. 5 illustrates a vaso-occlusive device 10 with the following loop pattern: larger OD loop; smaller OD loop; and smaller OD loop. The vaso-occlusive device 10 can be form by co-winding a first coil wire 16 having a larger OD with second and third coil wires 18, 22 each having a smaller OD. Alternatively, a larger OD first coil wire 16 can be co-wound with a smaller OD second coil wire 18, but two loops of the second coil wire 18 are formed for every loop of the first coil wire 16. FIG. 6 illustrates a similar vaso-occlusive device 10 with the following loop pattern: larger OD loop; smaller OD loop; smaller OD loop; and smaller OD loop. The vaso-occlusive device 10 can be formed by methods similar to those described for forming the vaso-occlusive device 10 of FIG. 5. Vaso-occlusive devices like those in FIGS. 5 and 6 have increased softness while maintaining bending performance similar to the vaso-occlusive device in FIG. 1. In particular, the vaso-occlusive devices in FIGS. 5 and 6 have more bending points along their length compared to the vaso-occlusive device in FIG. 1.

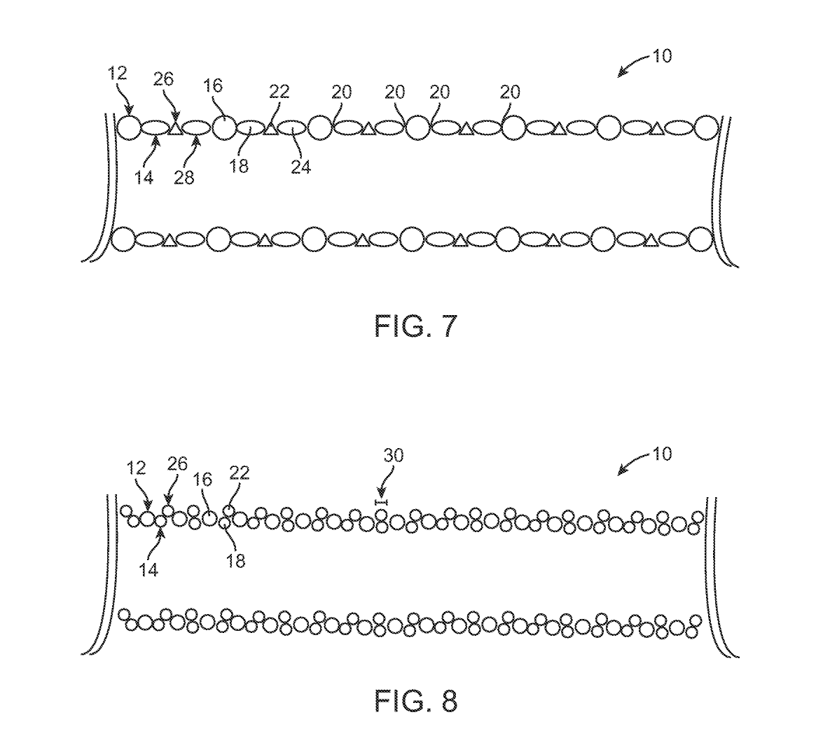

The vaso-occlusive device 10 depicted in FIG. 7 has loops of different ODs and cross-sectional geometries. The vaso-occlusive device 10 can be co-wound from first, second, third, and fourth coil wires 16, 18, 22, 24. The first coil wire 16 has a larger OD and a circular cross-sectional geometry. The second coil wire 18 and fourth coil wire 24 have the same medium OD and oval cross-sectional geometry. The third coil wire 22 has a smaller OD and triangular cross-sectional geometry. The first, second, third, and fourth coil wires 16, 18, 22, 24 may have different cross-sectional areas, particularly when they have different cross-sectional geometries.

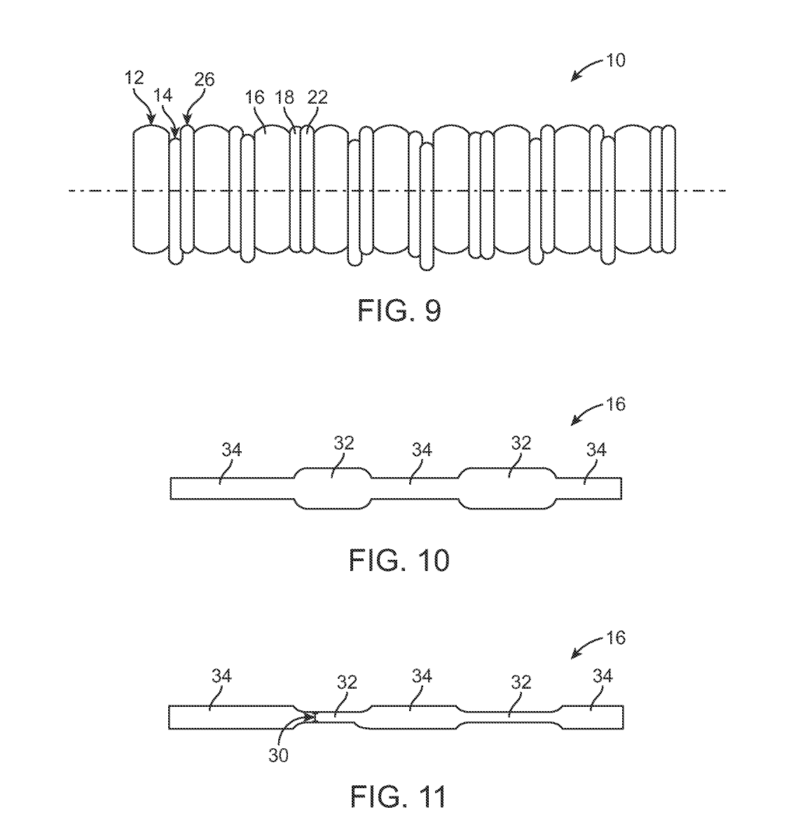

FIGS. 8 and 9 show a vaso-occlusive device 10 formed from a larger OD first coil wire 16 and smaller OD second and third coil wires 18, 22, which are pre-twisted together before co-winding with first coil wire 16. The pre-twisted pair of second and third coil wires 18, 22 has a defined pitch. The resulting vaso-occlusive device 10 has non-uniform bending behavior not only along its length, but also along its circumference. The vaso-occlusive device 10 is more likely to bend at a loop made from the pre-twisted pair of second and third coil wires 18, 22, which each have a smaller OD than the first coil wire 16. The vaso-occlusive device 10 is also more likely to bend along the short cross-sectional axis 30 of the pre-twisted pair of smaller OD second and third coil wires 18, 22 where the moment of inertia or bending stiffness is lowest.

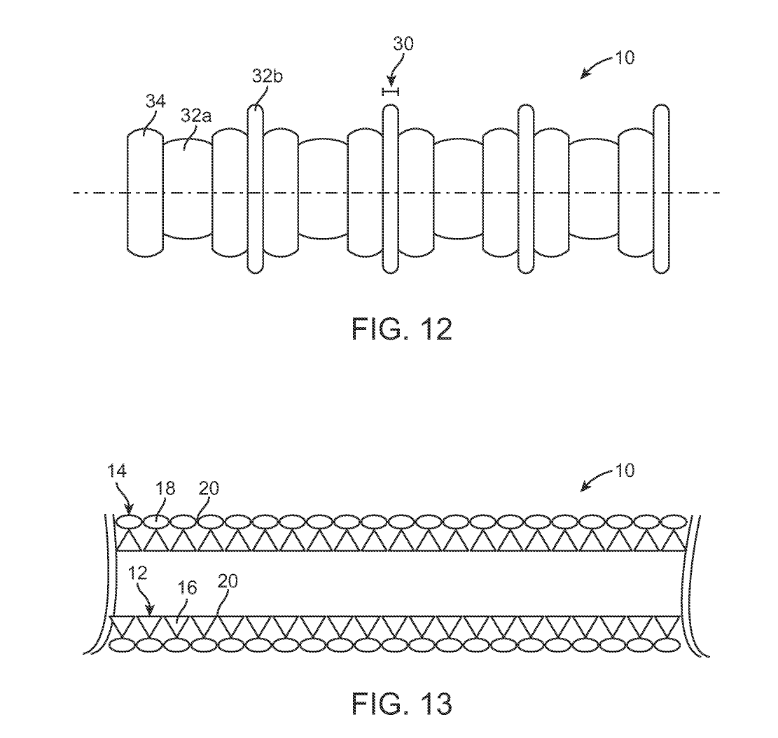

FIGS. 10 and 11 illustrate a coil wire 16 having flattened sections 32 with a short cross-sectional axis 30 (see FIG. 11). The vaso-occlusive device 10 in FIG. 12 is made from a coil wire 16 with three sections: an un-flattened wire section 34; a first flattened wire section 32a wound with the short cross-sectional axis 30 perpendicular to the longitudinal axis of the vaso-occlusive device 10; and a second flattened wire section 32b wound with the short cross-sectional axis 30 parallel to the longitudinal axis of the vaso-occlusive device 10. The vaso-occlusive device 10 is more likely to bend at the first flattened wire sections 32a. The un-flattened wire section 34 can have any cross-sectional geometry (i.e., round, oval, square, triangular, etc.)

Regarding the above-described embodiments of FIGS. 1-12, the wires 16, 18, 22, 24 forming the various coils 12, 14, 26, 28 may alternatively be made from a pure platinum, platinum-tungsten alloy, platinum-iridium alloy, platinum rhenium alloy, or platinum palladium alloy. The coils 12, 14, 26, 28 may also be made of wire with a platinum core with an outer layer of platinum-tungsten alloy, or from a material consisting of a core of platinum-tungsten alloy and an outer layer of platinum. Furthermore, the respective coils 12, 14, 26, 28 of embodiments of the presently disclosed inventions can alternatively be made of a biopolymer, a bioceramic, a bioactive material, or a combination of such materials. For example, a bioactive coating may be applied to any of the metallic, biopolymeric and/or bioceramic coils 12, 14, 26, 28.

It should be appreciated that the materials for forming the coils 12, 14, 26, 28 of the vaso-occlusive device 10 are not be limited to the examples described previously. In any of the embodiments described herein, the material for the coils 12, 14, 26, 28 may be a radio-opaque material such as a metal or a polymer. Also, in other embodiments, the material for the coils 12, 14, 26, 28 may be rhodium, palladium, rhenium, as well as tungsten, gold, silver, tantalum, and alloys of these metals. These metals have significant radio-opacity and in their alloys may be tailored to accomplish an appropriate blend of flexibility and stiffness. They are also largely biologically inert. Also, any materials which maintain their shape despite being subjected to high stress may be used to construct the coils 12, 14, 26, 28.

For example, certain “super-elastic alloys” include various nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum), may be used. In further embodiments, titanium-nickel alloy known as “nitinol” may be used to form the coils 12, 14, 26, 28. These are very sturdy alloys which will tolerate significant flexing without deformation even when used as very small diameter wire. Further, some or all of the wires 16, 18, 22, 24 may be made from different materials than the other wires.

In any of the embodiments described herein, the wires 16, 18, 22, 24 used to form the respective coils 12, 14, 26, 28 may have a cross-sectional dimension that is in the range of 0.00002 and 0.01 inches. The coils 12, 14, 26, 28 formed by the respective wires 16, 18, 22, 24 may have a cross-sectional dimension between 0.003 and 0.03 inches. In various embodiments, the wires 16, 18, 22, 24 can have any geometry, such as square, rectangle, or circle. For neurovascular applications, the diameter of the coils 12, 14, 26, 28 may be anywhere from 0.008 to 0.018 inches. In other embodiments, the wires 16, 18, 22, 24 may have other cross-sectional dimensions, and the coils 12, 14, 26, 28 may have other cross-sectional dimensions. In some embodiments, the wires 16, 18, 22, 24 for forming the coils 12, 14, 26, 28 should have a sufficient diameter to provide a hoop strength to the resulting vaso-occlusive coil 10 sufficient to hold the coil 10 in place within the chosen body site, lumen or cavity, without substantially distending the wall of the site and without moving from the site as a result of the repetitive fluid pulsing found in the vascular system.

In any of the embodiments described herein, the axial length of the coils 12, 14, 26, 28 may be in the range of 0.5 to 100 cm, and more preferably, in the range of 2.0 to 40 cm. Depending upon use, the coils 12, 14, 26, 28 may have 10-75 turns per centimeter, or more preferably 10-40 turns per centimeter. In other embodiments, the coils 12, 14, 26, 28 may have other lengths and/or other number of turns per centimeter.

Further, while the above-described embodiments of FIGS. 1-12 are directed to single layer coils, it should be appreciated by those skilled in the art that double-coil embodiments, i.e., having an outer coil layer and an inner coil layer may be included in alternative embodiments, in accordance with the inventive aspects disclosed herein.

FIG. 13 illustrates a vaso-occlusive device 10 in accordance with an alternate embodiment, wherein the vaso-occlusive device 10 has a first inner coil 12 and a second outer coil 14 disposed around the first inner coil 12. The first inner coil 12 has a triangular cross-sectional geometry and the second outer coil 14 has an oval cross-sectional geometry. Further, the first coil wire 16 and the second coil wire 18 may have different cross-sectional areas, particularly when they have different cross-sectional geometries. The first inner coil 12, the second outer coil 12, or both may be made from a plurality of outer coil wires that are twisted together as described above. Moreover, the first inner coil 12, the second outer coil 12, or both may include an un-flattened section and a flattened section as described above. In addition, one of the first inner coil 12 and the second outer coil 14 can be wound in a clockwise direction and the other of the first inner coil 12 and the second outer coil 14 can be wound in a counter-clockwise direction. Further, one or more additional coil layers may be included in alternative embodiments for a total of three or more coil layers, in accordance with the inventive aspects disclosed herein. Such three-or-more coil layer embodiments would comprise an outer coil layer, and two or more inner coil layers.

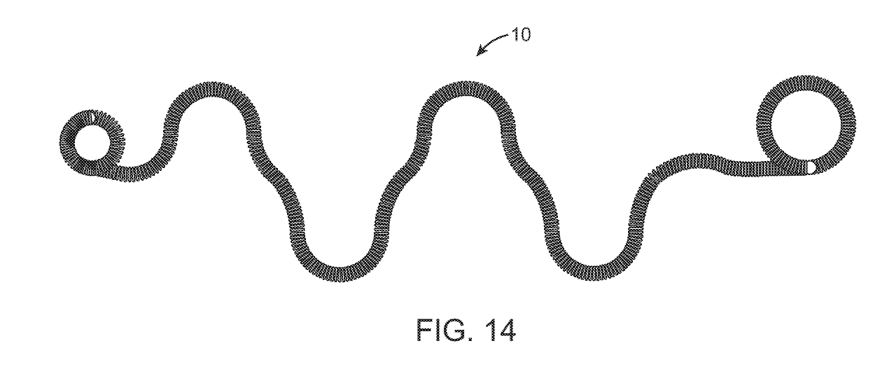

In some embodiments, the vaso-occlusive devices 10 described herein may have the simple linear shape shown previously, or may have shapes which are more complex. FIG. 14 shows what is termed a “secondary” shape in that it is formed from the primary coil by winding the primary coil on a form of a desired shape, e.g. a mandrel, and then heat treating the so-formed shape. Various other secondary shapes may be implemented in embodiments of the vaso-occlusive device 10 described herein.

Although particular embodiments have been shown and described herein, it will be understood by those skilled in the art that they are not intended to limit the present inventions, and it will be obvious to those skilled in the art that various changes and modifications may be made (e.g., the dimensions of various parts) without departing from the scope of the disclosed inventions, which is to be defined only by the following claims and their equivalents. The specification and drawings are, accordingly, to be regarded in an illustrative rather than restrictive sense. The various embodiments shown and described herein are intended to cover alternatives, modifications, and equivalents of the disclosed inventions, which may be included within the scope of the appended claims.

What is claimed is:

1. A vaso-occlusive device, comprising:
a first wire having a first cross-sectional geometry and
a second wire having a second cross-sectional geometry, wherein the first cross-sectional geometry is different from the second cross-sectional geometry.

2. The vaso-occlusive device of claim 1, wherein the first wire is wound to form a first coil,
wherein the second wire is wound to form a second coil defining a lumen therein, and
wherein the first coil is disposed at least partially in the lumen.

3. The vaso-occlusive device of claim 2, wherein the first wire has a first cross-sectional area,
wherein the second wire having a second cross-sectional area, and
wherein the first cross-sectional area is different from the second cross-sectional area.

4. The vaso-occlusive device of claim 2, wherein the first wire is made from a first material,
wherein the second wire is made from a second material, and
wherein the first material is different from the second material.

5. The vaso-occlusive device of claim 2, wherein at least one of the first and second wires is made from a plurality of wires that are twisted together.

6. The vaso-occlusive device of claim 2, wherein one of the first and second coils is wound in a clockwise direction and the other of the first and second coils is wound in a counter-clockwise direction.

7. The vaso-occlusive device of claim 1, wherein the first wire and the second wire are co-wound to form a single coil.

8. The vaso-occlusive device of claim 7, wherein the first wire has a first cross-sectional area,
wherein the second wire having a second cross-sectional area, and
wherein the first cross-sectional area is different from the second cross-sectional area.

9. The vaso-occlusive device of claim 7, wherein the first wire is made from a first material,
wherein the second wire is made from a second material, and
wherein the first material is different from the second material.

10. The vaso-occlusive device of claim 7, wherein at least one of the first and second wires is made from a plurality of wires that are twisted together.

11. The vaso-occlusive device of claim 7, wherein the second wire forms more than one loop for each loop formed by the first wire.

12. The vaso-occlusive device of claim 11, further comprising discrete contact points between the first coil and the second coil.

13. The vaso-occlusive device of claim 11, further comprising a third wire at least partially co-wound with the first and second wires.

14. A vaso-occlusive device comprising a coil wire having an un-flattened section, a first flattened section, and a second flattened section,
wherein a short cross-sectional axis of the first flattened section lies substantially perpendicular to a longitudinal axis of the vaso-occlusive device, and a short cross-sectional axis of the second flattened section lies substantially parallel to the longitudinal axis of the vaso-occlusive device.

15. The vaso-occlusive device of any of claims 14, wherein the un-flattened section of the coil wire has a triangular cross-sectional geometry.
